(19) United States
(12) Patent Application Publication
(10) Pub. No.: US 2005/0266018 A1
(43) Pub. Date: Dec. 1, 2005

(54) Nutraceutical compositions with mangosteen

(76) Inventors: Benson K. Boreyko, Scottsdale, AZ (US); Yibing Wang, Gilbert, AZ (US)

Correspondence Address: QUARLES & BRADY LLP, RENAISSANCE ONE, TWO NORTH CENTRAL AVENUE, PHOENIX, AZ, 85004-2391, US

(21) Appl. No.: 10/857,612

(22) Filed: May 27, 2004

(51) Int. Cl. A61K 35/78; A61K 35/80

(52) U.S. Cl. 424/195.17; 424/729; 424/744; 424/769

(57) ABSTRACT

A nutraceutical composition contains mangosteen juice, mangosteen pericarp and aloe. Another composition contains mangosteen juice, mangosteen pericarp and tea. Optionally these compositions can include additional components such as algae, fungi such as mushrooms, cacti, vitamins, minerals, herbs, fruits and/or vegetables, and flavors.

### TECHNICAL FIELD

The present invention relates to nutraceutical compositions derived from the fruit of the Garcinia mangostana L. plant, otherwise known as the mangosteen plant. More particularly, the present invention relates to nutraceutical compositions comprising a mixture of the pulp and pericarp (rind) of the Mangosteen fruit combined with Aloe vera juice.

### BACKGROUND

Mangosteen fruit has long been used in India and China for medicinal purposes. The sliced and dried rind (pericarp) has been powdered and administered to those with dysentery. Made into an ointment, it is applied on eczema and other skin disorders. The rind decoction is taken to relieve diarrhea and cystitis, gonorrhea and gleet and is applied externally as an astringent lotion. A portion of the rind is steeped in water overnight and the infusion is given as a remedy for chronic diarrhea in adults and children. The rind of partially ripe fruits yields a polyhydroxy-xanthone derivative termed mangostin, also B-mangostin. That of fully ripe fruits contains the xanthones, gartanin, 8-disoxygartanin and normangostin. A derivative of mangostin, mangostin-e, 6-di-O-glucoside, is a central nervous system depressant and causes a rise in blood pressure.

Mangostin (M) and its derivatives such as 3-O-methyl mangostin (MM), mangostin 3,6-di-O-methyl mangostin (DM), 1-isomangostin (IM), mangostin triacetate (MT), mangostin 3,6-di-O-(tetra acetyl) glucoside (MTG) and mangostin-6,6-di-O-glucoside (MOG) have been screened for pharmacological effects in experimental animals. All but DM produced central nervous system depression characterized by ptosis, sedation and decreased motor activity. Only MOG produced significant effects on the cardiovascular system, namely myocardial stimulation and a rise in blood pressure. M, IM and MT produced pronounced anti-inflammatory activity both by intraperitoneal and oral routes. M alone produced significant anti-ulcer activity in rats.

Mangosteen, Garcinia mangostana Linn. (Guttiferae), is one of the most praised tropical fruits. The Mangosteen tree is very slow-growing, erect, with a pyramidal crown. It attains 20-80 ft in height. Its bark is dark-brown or nearly black and flaking. The evergreen opposite, short-stalked leaves are ovate-oblong or elliptic, leathery and thick, dark-green, slightly glossy above, yellowish-green and dull beneath. The flowers are 1-½ to 2 inches wide and fleshy and may be male or hermaphrodite on the same tree.

The gel of the Aloe vera plant (Aloe barbadensis) has a long history as a medicinal plant to treat burns, skin inflammation, etc. One of Aloe Vera's active ingredients is the complex carbohydrate called acemannan. This ingredient has been shown to be an effective treatment for several forms of viruses and also enhances the capabilities of the immune system. Acemannan has been shown to activate several types of white blood cells within the immune system, increasing amounts of tumor necrosis factor, gamma interferon and interleukin 1, all of which increase the body's ability to destroy viruses, bacteria and tumor cells.

Aloe, native to Africa, is also known as the lily of the desert, the plant of immortality and the medicine plant. Its name derived from the Arabic “alloeh” meaning bitter, because of the bitter liquid found in the leaves. The plant is about 96% water. The rest of it contains active ingredients including essential oils, amino acids, minerals, vitamins, enzymes and glycoproteins. It is used in many liquid health treatments, sometimes in combination with other plants and herbs. The juice is soothing to digestive tract irritations, such as colitis and peptic ulcers. It is also said to facilitate digestion, aid in blood and lymphatic circulation, as well as kidney, liver and gall bladder functions. Its bitter flavor denotes alkalinity which balances the digestive acid and may be helpful for the stomach, small intestine and colon. Some have used Aloe juice for peptic ulcers and for gastrointestinal health. Because Aloe vera is so prized for its medicinal value and its flavor is bitter, it is not generally ingested as a mere fruit or vegetable.

Aloe vera gel is the leaf pulp or mucilage, a thin clear jelly-like substance obtained from the tissue of the inner portion of the leaves. Aloe latex, commonly referred to as aloe juice, is a bitter yellow exudate from the pericyclic tubules just beneath the outer skin of the leaves and has been used as a laxative. Its laxative effect has been attributed to the presence of anthraquinone glycosides aloin A and B, which are potent laxatives. However, aloe products for internal use have been promoted not just for constipation, but also for coughs, wounds, ulcers, diabetes, cancer, headaches, arthritis, immune-system deficiencies, and many other conditions.

What is needed is a tonic beverage to deliver the benefits of the mangosteen fruit pulp with pericarp in a mixture with a high free radical absorption capacity.

### SUMMARY OF THE INVENTION

It is an object of this invention to provide a product with a high capacity to absorb free radicals.

In one embodiment, there is provided a nutraceutical composition containing mangosteen juice, mangosteen pericarp and Aloe vera. Optionally, the composition has at least one vitamin. The vitamin can be selected from Vitamin A, Vitamin C, Vitamin D, Vitamin E, Vitamins B6 and B12, biotin, niacin, pantothenic acid, thiamin, riboflavin, folic acid and a combination thereof. In addition, the nutraceutical composition may contain at least one type of alga. Algae such as blue green alga, red alga, brown alga or chlorella can be used. One particular alga is spirulina.

In another embodiment, the nutraceutical composition of mangosteen juice, mangosteen pericarp and Aloe vera also may contain at least one type of tea obtained from leaves, crushed leaves, other plant parts, infusions thereof, or extractions thereof. Examples of tea sources include green tea leaves, black tea leaves, one or more herbs, or a combination thereof. Alternately, the composition may contain an edible cactus, such as an opuntia, a nopalea or a combination thereof. The edible cactus can be in the form of a leaf, fruit, juice, concentrate or extract.

In another embodiment, the nutraceutical composition of mangosteen juice, mangosteen pericarp and Aloe vera also may contain one or more fungi, such as a mushroom. Such mushrooms include maitake, shiitake, reishi, or a combination thereof. Added to the nutraceutical composition can be one or more herbs, such as angelica, basil, calendula, chamomile, yarrow, coriander, feverfew, horehound, hyssop, echinacea, St. John's wort, summer savory, thyme, ginseng, kava, pennyroyal, valerian or a combination thereof. Another possible component includes a fruit and/or a vegetable. The fruit or vegetable can be provided as a juice, juice concentrate, puree or extract. Examples include alfalfa, apple, apricot, banana, beet, blackberry, blueberry, cantaloupe, carrot, celery, cherry, cranberry, grape, grapefruit, green barley, lemon, green lettuce, kale, kiwi, mango, orange, papaya, parsley, peach, pear, pineapple, plum, prune, raspberry, spinach, strawberry, tangerine, tomato, watermelon, wolfberry or a combination thereof. The inventive combination can also include a mineral, such as selenium.

In another embodiment, there is provided a nutraceutical composition with mangosteen juice, mangosteen pericarp and tea. This composition additionally comprises at least one vitamin. One or more vitamins can be selected from Vitamin A, Vitamin C, Vitamin D, Vitamin E, Vitamins B6 and B 12, biotin, niacin, pantothenic acid, thiamin, riboflavin, folic acid and a combination thereof. Optionally, this composition can contain at least one type of alga, such as blue green alga, red alga, brown algae or a combination thereof. A particularly preferred alga is spirulina. The tea can be obtained from leaves, crushed leaves, other plant parts, infusions thereof, or extractions thereof. The tea can be obtained from green tea leaves, black tea leaves, one or more herbs or a combination thereof.

In another embodiment, the nutraceutical composition of mangosteen juice, mangosteen pericarp and tea can be combined with an edible cactus, such as an opuntia, a nopalea or a combination thereof. The edible cactus is in the form of a leaf, fruit, juice, concentrate or extract. Optionally the nutraceutical composition also may contain one or more fungi, particularly a mushroom, such as maitake, shiitake, reishi, or a combination thereof. Another option is the inclusion of one or more herbs, such as angelica, basil, calendula, chamomile, yarrow, coriander, feverfew, horehound, hyssop, echinacea, St. John's wort, summer savory, thyme, ginseng, kava, pennyroyal, valerian or a combination thereof. Optionally, the nutraceutical composition can contain a fruit and/or a vegetable, which may be provided as a juice, juice concentrate, puree or extract. Examples include alfalfa, apple, apricot, banana, beet, blackberry, blueberry, cantaloupe, carrot, celery, cherry, cranberry, grape, grapefruit, green barley, lemon, green lettuce, kale, kiwi, mango, orange, papaya, parsley, peach, pear, pineapple, plum, prune, raspberry, spinach, strawberry, tangerine, tomato, watermelon, wolfberry or a combination thereof. The inventive combination can also include a mineral, such as selenium.

In another embodiment, there is provided a nutraceutical composition including mangosteen juice, mangosteen pericarp, Aloe vera juice and/or gel, a vitamin blend, fructose, green tea, a pH adjuster, preservatives such as sodium benzoate and potassium, a thickener such as xanthan gum, selenium, and orange essential oils, wherein the vitamin blend includes Vitamins A, C, D, E, B6 and B12, thiamine, riboflavin, niacin, folate, biotin and pantothenic acid, and the pH adjuster is malic acid.

In another embodiment, there is a nutraceutical composition including mangosteen juice, mangosteen pericarp, Aloe vera juice and/or gel, a vitamin blend, fructose, fruit and vegetable juices, orange essential oils, raspberry flavor, citric acid, selenium, a thickener such as xanthan gum and preservatives, wherein the preservatives are sodium benzoate and potassium sorbate. In this embodiment, the fruit and vegetable juices can be selected from apple juice, pear juice, grape juice, blueberry juice, raspberry juice, strawberry juice, cranberry juice, cherry juice, fruit blend, wolfberry juice, carrot juice, tomato juice, beet juice and a combination thereof.

In another embodiment, there is a nutraceutical composition comprising mangosteen juice, mangosteen pericarp and alga. Optionally, the composition has at least one vitamin. The vitamin can be selected from Vitamin A, Vitamin C, Vitamin D, Vitamin E, Vitamins B6 and B12, biotin, niacin, pantothenic acid, thiamin, riboflavin, folic acid and a combination thereof. Algae such as blue green alga, red alga, brown alga or chlorella can be used. One particular alga is spirulina. Alternately, the composition may contain an edible cactus, such as an opuntia, a nopalea or a combination thereof. The edible cactus can be in the form of a leaf, fruit, juice, concentrate or extract. The inventive composition also may contain one or more fungi, such as a mushroom. Such mushrooms include maitake, shiitake, reishi, or a combination thereof. Added to the nutraceutical composition can be one or more herbs, such as angelica, basil, calendula, chamomile, yarrow, coriander, feverfew, horehound, hyssop, echinacea, St. John's wort, summer savory, thyme, ginseng, kava, pennyroyal, valerian or a combination thereof. Another possible component includes a fruit and/or a vegetable. The fruit or vegetable can be provided as ajuice, juice concentrate, puree or extract. Examples include alfalfa, apple, apricot, banana, beet, blackberry, blueberry, cantaloupe, carrot, celery, cherry, cranberry, grape, grapefruit, green barley, lemon, green lettuce, kale, kiwi, mango, orange, papaya, parsley, peach, pear, pineapple, plum, prune, raspberry, spinach, strawberry, tangerine, tomato, watermelon, wolfberry or a combination thereof.

In another embodiment, there is a nutraceutical composition comprising mangosteen juice, mangosteen pericarp and edible cactus. Optionally, the composition has at least one vitamin. The vitamin can be selected from Vitamin A, Vitamin C, Vitamin D, Vitamin E, Vitamins B6 and B12, biotin, niacin, pantothenic acid, thiamin, riboflavin, folic acid and a combination thereof. In addition, the nutraceutical composition may contain at least one type of alga. Algae such as blue green alga, red alga, brown alga or chlorella can be used. One particular alga is spirulina. The edible cactus can be an opuntia, a nopalea or a combination thereof. The edible cactus can be in the form of a leaf, fruit, juice, concentrate or extract. It also may contain one or more fungi, such as a mushroom. Such mushrooms include maitake, shiitake, reishi, or a combination thereof. Added to the nutraceutical composition can be one or more herbs, such as angelica, basil, calendula, chamomile, yarrow, coriander, feverfew, horehound, hyssop, echinacea, St. John's wort, summer savory, thyme, ginseng, kava, pennyroyal, valerian or a combination thereof. Another possible component includes a fruit and/or a vegetable. The fruit or vegetable can be provided as a juice, juice concentrate, puree or extract. Examples include alfalfa, apple, apricot, banana, beet, blackberry, blueberry, cantaloupe, carrot, celery, cherry, cranberry, grape, grapefruit, green barley, lemon, green lettuce, kale, kiwi, mango, orange, papaya, parsley, peach, pear, pineapple, plum, prune, raspberry, spinach, strawberry, tangerine, tomato, watermelon, wolfberry or a combination thereof.

In another embodiment, there is a nutraceutical composition comprising mangosteen juice, mangosteen pericarp and fungi, particularly mushrooms. Optionally, the composition has at least one vitamin. The vitamin can be selected from Vitamin A, Vitamin C, Vitamin D, Vitamin E, Vitamins B6 and B12, biotin, niacin, pantothenic acid, thiamin, riboflavin, folic acid and a combination thereof. In addition, the nutraceutical composition may contain at least one type of alga. Algae such as blue green alga, red alga, brown alga or chlorella can be used. One particular alga is spirulina. Alternately, the composition may contain an edible cactus, such as an opuntia, a nopalea or a combination thereof. The edible cactus can be in the form of a leaf, fruit, juice, concentrate or extract. The mushroom can be maitake, shiitake, reishi, or a combination thereof. Added to the nutraceutical composition can be one or more herbs, such as angelica, basil, calendula, chamomile, yarrow, coriander, feverfew, horehound, hyssop, echinacea, St. John's wort, summer savory, thyme, ginseng, kava, pennyroyal, valerian or a combination thereof. Another possible component includes a fruit and/or a vegetable. The fruit or vegetable can be provided as a juice, juice concentrate, puree or extract. Examples include alfalfa, apple, apricot, banana, beet, blackberry, blueberry, cantaloupe, carrot, celery, cherry, cranberry, grape, grapefruit, green barley, lemon, green lettuce, kale, kiwi, mango, orange, papaya, parsley, peach, pear, pineapple, plum, prune, raspberry, spinach, strawberry, tangerine, tomato, watermelon, wolfberry or a combination thereof.

In another embodiment, there is a nutraceutical composition comprising mangosteen juice, mangosteen pericarp and at least one herb. Optionally, the composition has at least one vitamin. The vitamin can be selected from Vitamin A, Vitamin C, Vitamin D, Vitamin E, Vitamins B6 and B12, biotin, niacin, pantothenic acid, thiamin, riboflavin, folic acid and a combination thereof. In addition, the nutraceutical composition may contain at least one type of alga. Algae such as blue green alga, red alga, brown alga or chlorella can be used. One particular alga is spirulina. Alternately, the composition may contain an edible cactus, such as an opuntia, a nopalea or a combination thereof. The edible cactus can be in the form of a leaf, fruit, juice, concentrate or extract. It also may contain one or more fungi, such as a mushroom. Such mushrooms include maitake, shiitake, reishi, or a combination thereof. Examples of herbs include but are not limited to angelica, basil, calendula, chamomile, yarrow, coriander, feverfew, horehound, hyssop, echinacea, St. John's wort, summer savory, thyme, ginseng, kava, pennyroyal, valerian or a combination thereof. Another possible component includes a fruit and/or a vegetable. The fruit or vegetable can be provided as ajuice, juice concentrate, puree or extract. Examples include alfalfa, apple, apricot, banana, beet, blackberry, blueberry, cantaloupe, carrot, celery, cherry, cranberry, grape, grapefruit, green barley, lemon, green lettuce, kale, kiwi, mango, orange, papaya, parsley, peach, pear, pineapple, plum, prune, raspberry, spinach, strawberry, tangerine, tomato, watermelon, wolfberry or a combination thereof.

In another embodiment, there is a nutraceutical composition comprising mangosteen juice, mangosteen pericarp and at least one vitamin. The vitamin can be selected from Vitamin A, Vitamin C, Vitamin D, Vitamin E, Vitamins B6 and B12, biotin, niacin, pantothenic acid, thiamin, riboflavin, folic acid and a combination thereof. In addition, the nutraceutical composition may contain at least one type of alga. Algae such as blue green alga, red alga, brown alga or chlorella can be used. One particular alga is spirulina. Alternately, the composition may contain an edible cactus, such as an opuntia, a nopalea or a combination thereof. The edible cactus can be in the form of a leaf, fruit, juice, concentrate or extract. It also may contain one or more fungi, such as a mushroom. Such mushrooms include maitake, shiitake, reishi, or a combination thereof. Added to the nutraceutical composition can be one or more herbs, such as angelica, basil, calendula, chamomile, yarrow, coriander, feverfew, horehound, hyssop, echinacea, St. John's wort, summer savory, thyme, ginseng, kava, pennyroyal, valerian or a combination thereof. Another possible component includes a fruit and/or a vegetable. The fruit or vegetable can be provided as ajuice, juice concentrate, puree or extract. Examples include alfalfa, apple, apricot, banana, beet, blackberry, blueberry, cantaloupe, carrot, celery, cherry, cranberry, grape, grapefruit, green barley, lemon, green lettuce, kale, kiwi, mango, orange, papaya, parsley, peach, pear, pineapple, plum, prune, raspberry, spinach, strawberry, tangerine, tomato, watermelon, wolfberry or a combination thereof.

In another embodiment, there is a nutraceutical composition comprising mangosteen juice, mangosteen pericarp and extracts of at least one fruit or vegetable. Optionally, the composition has at least one vitamin. The vitamin can be selected from Vitamin A, Vitamin C, Vitamin D, Vitamin E, Vitamins B6 and B12, biotin, niacin, pantothenic acid, thiamin, riboflavin, folic acid and a combination thereof. In addition, the nutraceutical composition may contain at least one type of alga. Algae such as blue green alga, red alga, brown alga or chlorella can be used. One particular alga is spirulina. Alternately, the composition may contain an edible cactus, such as an opuntia, a nopalea or a combination thereof. The edible cactus can be in the form of a leaf, fruit, juice, concentrate or extract. It also may contain one or more fungi, such as a mushroom. Such mushrooms include maitake, shiitake, reishi, or a combination thereof. Added to the nutraceutical composition can be one or more herbs, such as angelica, basil, calendula, chamomile, yarrow, coriander, feverfew, horehound, hyssop, echinacea, St. John's wort, summer savory, thyme, ginseng, kava, pennyroyal, valerian or a combination thereof. The fruit or vegetable can be provided as a juice, juice concentrate, puree or extract. Examples include alfalfa, apple, apricot, banana, beet, blackberry, blueberry, cantaloupe, carrot, celery, cherry, cranberry, grape, grapefruit, green barley, lemon, green lettuce, kale, kiwi, mango, orange, papaya, parsley, peach, pear, pineapple, plum, prune, raspberry, spinach, strawberry, tangerine, tomato, watermelon, wolfberry or a combination thereof.

In another embodiment, there is a nutraceutical composition comprising a mangosteen extract selected from whole mangosteen, mangosteen fruit juice, mangosteen pericarp, mangosteen pericarp juice or a combination thereof. Optionally, the nutraceutical composition includes at least one fruit or vegetable juice, puree or extract. Optionally, the nutraceutical composition includes an aloe product obtained from whole leaf, leaf cuticle, leaf gel, an extract thereof, or a combination thereof. Optionally, the nutraceutical composition includes an alga such as spirulina. Optionally, the nutraceutical composition includes a tea. Optionally, the nutraceutical composition includes at least one edible cactus. Optionally, the nutraceutical composition includes at least one edible fungi or mushroom. Optionally, the nutraceutical composition includes at least one herb. Optionally, the nutraceutical composition includes at least one vitamin. Optionally, the nutraceutical composition includes at least one mineral.

### DETAILED DESCRIPTION

The combination of mangosteen and selected substances provides a powerful plant-based nutraceutical composition. Mangosteen provides super-concentrated antioxidant protection from whole fruit juices and features a full array of phytonutrients including the important xanthones. To maximize the xanthone and antioxidant content, the whole fruit of the mangosteen, including the xanthone-rich rind (pericarp) is used.

The components in the inventive combination can be obtained from natural sources or chemical synthesis. When obtained from natural sources, the source can be organic. Preparation of the components from the natural sources commonly include but are not limited to extraction or evaporation (concentration). Extracts can be prepared routinely, e.g., by contacting the plant parts with a suitable solvent to extract a phytochemical or other compound from the material (e.g., see, U.S. Pat. No. 4,118,508, JP 11292777, JP 6133731 and PCT Publication WO 00/72861 by Martin, for extraction processes).

The inventive composition has an outstanding ORAC score. The test, called oxygen radical absorbance capacity, measures the total antioxidant potency of foods or supplements. It is a more precise way of determining the free radical-destroying power of a food than just focusing on individual nutrients, because ORAC takes into consideration the effect of all of the plant's compounds, including many phytochemicals that are not traditionally considered nutrients, and the impact they have when they work in concert. Very simply, a sample of a food or a chemical substance is put in a test tube to measure how well and for how long it disarms free radicals. The test substance is then given an ORAC score that reflects its free radical neutralizing power.

Whole leaf Aloe vera provides not only the internal gel but also the yellow latex of the leaf cuticle (outer layer). Aloe vera extracts can eliminate dozens of different types of harmful bacteria. One of Aloe Vera's most important active ingredients is the complex carbohydrate known as acemannan. This ingredient has been shown to be an effective treatment for several forms of viruses, and it also enhances the capabilities of the immune system. Acemannan's exact mode of action is not yet fully understood. Research shows, however, that it activates several types of white blood cells within the immune system. It appears to increase amounts of tumor necrosis factor, gamma interferon and interleukin 1, all of which increase the body's ability to destroy viruses, bacteria and tumor cells.

Acemannan is effective in treatment of feline leukemia. This disease is attributed to a retrovirus and is usually lethal: Generally about 40% of infected cats die within four weeks of exhibiting clinical symptoms of the disease and 70% within eight weeks. Nevertheless, several studies have shown that acemannan dramatically changed the course of this disease. For example, in one trial 49 cats were divided into three groups and given acemannan for a 12-week period. The three groups received the acemannan intravenously, orally or by injection. Thirteen cats (26%) died during the 12-week period. Remarkably, 36 of the 49 (74%) were alive and survived five to 19 months following the start of the study.

Another study suggests that acemannan could be an adjunctive treatment for cancer. Forty-three cats and dogs with spontaneous tumors were administered acemannan by injection. In 26 of the animals (60%), the tumors showed distinct positive changes. Twelve of these animals had obvious shrinkage, encapsulation, and liquefaction or actual death of the tumor.

Acemannan by itself is considered a drug and currently is approved in the United States for veterinary use only. The USDA has granted the manufacturer a license to market the injectable drug for the treatment of feline leukemia in cats and fibrosarcomas in cats and dogs. It is considered safe and generally without side effects.

In addition, Aloe vera contains at least three anti-inflammatory fatty acids that are helpful for the stomach, small intestine and colon. Recently, Moon EJ (Angiogenesis 1999 3(2): 117-23) proved that Aloe vera fatty acid beta-sitosterol has an angiogenic effect (stimulating blood vessel growth) that would be beneficial in wound healing. Furthermore, Lim BO et al. (J. Nutr. Sci. Vitaminol. (Tokyo) 2003 49(4): 292-6) demonstrated in a longitudinal study of rats that cholesterol levels were 30% lower and there was less free radical-induced oxidative damage than in controls not receiving Aloe vera.

The Aloe vera latex contains the anthraquinone glycosides aloin A and B, which are potent laxatives. They increase colonic peristalsis and possibly increase intestinal water. These effects result in somewhat more frequent stools with softer consistency. Administration of Aloe vera also has been shown to decrease fasting blood sugar in diabetic animals (e.g., Ghannam N. Hormone Research 1986 24:288-94).

The latex of Aloe vera also has been shown to be effective against bacteria such as Corynebacterium, Salmonella, Streptococcus and Staphylococcus aureus (Martinez MJ et al. Ethnopharmacol. 1996, 51:171-74). An extract of Aloe barbadensis inactivated a number of different viruses, as did purified compounds obtained therefrom (Sydiskis RJ et al. Antimicrob Agents Chemother 1991 35(12): 2463-66). The glycoprotein verectin obtained from Aloe vera has been shown to be a COX-2 inhibitor (Yagi A. et al Planta Med. 2003 69(3): 269-71). COX-2 inhibitors are commonly used in inflammatory conditions such as arthritis. Two important pathogens, Shigella flexneri (a cause of watery diarrhea) and Streptococcus pyogenes (a cause of toxic shock syndrome), have become resistant to antibiotic therapy but are still susceptible to Aloe vera (Ferro VA et al. Antimicrob. Agents Chemother. 2003 47(3): 1137-39).

There are also many optional factors which can be used in the inventive combination with mangosteen and/or Aloe vera. Teas of various types are important nutritionals. Other optional components include fungus, algae, vitamins, fruits, vegetables, cacti and herbs. Teas are typically made by steeping parts of plants in hot water for several minutes. Generally the leaves are used; however, other parts, including but not limited to stems, flowers and flower petals, and roots may be used, depending on the plant, the harvest season and the desired effect. The main types of teas are black tea, green tea, oolong, jasmine tea and herbal teas, the latter of which includes as many different plants as the imagination can employ. Moderate tea consumption, defined in the study as two cups a week, was associated with a 28 percent lower death rate when compared to the death rate of non-drinkers. Heavy tea drinkers, who averaged 19 cups a week, fared even better: They had a 44 percent lower death rate than non-drinkers during the four-year follow up, even though the non-drinkers' ages averaged two years less. The tea's flavonoids, powerful antioxidants, help improve the blood vessels' ability to relax. Flavonoids also prevent the so-called bad cholesterol, or “LDL cholesterol,” from oxidizing, which experts believe may promote hardening of the arteries. The substances may also keep blood from clotting too much.

Green tea has been known for centuries and has been studied by scientists for at least a century. Green tea extract has reduced the incidence of cancer in mice inoculated with cancer cells and given carcinogenic chemicals. A major ingredient which has been studied intensively is catechin (the main ingredient of green tea tannin). Moreover, inhabitants of a Japan province where green tea is the staple product and the main beverage have a significantly lower risk of stomach cancer than Japanese outside tea-growing areas. Green tea catechin fed to rats on a high fat diet that produced excessive LDL and cholesterol levels in control rats prevented increased LDL cholesterol with minimal effect on the amount of HDL cholesterol (Muramatsu K. J. Nutr. Sci. Vitaminol. 1986 32:613). Green tea catechin also inhibited the angiotensin converting enzyme (ACE) and thus prevented age-related increases in blood pressure (Hara Y. et al. Nippon Nogeikagaku Kaishi 1987 61: 803). Green tea also has been observed to decrease the blood sugar in diabetics, increase alertness (through its caffeine content), kill bacteria, treat diarrhea, and stop dental cavities (by killing the cariogenic bacteria and blocking the plaque-building enzyme) It even may suppress aging (e.g., development of the aging pigment lipofuscin) by its antioxidant action. A recent study at the Institute of Physiology at the University of Fribourg in Switzerland revealed that green tea extract boosts the burning of calories and fat needed to lose weight. Green tea did this by increasing thermogenesis, the generation of body heat that naturally occurs as a result of the digestion, absorption and metabolization of food.

Black tea also has been endowed with significant health benefits. Regular consumption of black tea may protect against a variety of cancers. It has recently been found to contain theaflavin-3′-monogallate (TF-2), that in the laboratory allowed normal cells to flourish while cancer cells died. TF-2 also suppresses COX-2 activity and inflammation. A study involving people diagnosed with coronary artery disease reported that subjects who drank four cups of black tea per day showed improved blood vessel function. These results suggest that drinking tea may improve an important underlying abnormality of blood vessel function that may be related to coronary artery disease.

Other sources of herbal tea include but are not limited to persimmon, sassafras, birch, blackberry, raspberry, blueberry, alfalfa, strawberry, rose hips, peppermint, angelica, sweet basil, calendula, catnip, chamomile, common yarrow, coriander, feverfew, horehound, hyssop, purple coneflower (Echinacea purpurea), St. John's wort, summer savory, thyme and valerian.

Algae are simple organisms but are also a rich source of minerals, trace minerals and rare earth elements. Some names of common edible seaweed algae include but are not limited to nori, wakame, arame, dulse, kelp, wakame and Irish moss. They are known to be rich in iodine, calcium, iron, silicon, copper, zinc and selenium. Micro-algae, or single-celled algae include but are not limited to spirulina, chlorella and wild blue-green algae. In the dried form, they are rich in proteins, beta-carotene, nucleic acids (RNA and DNA) and gamma-linolenic acid (GLA). GLA is an essential fatty acid needed to make vital prostaglandins that reduce blood platelet adhesion and unwanted blood clotting.

Micro-algae have been said to improve immunity of people with cancer, AIDS, colitis and diabetes. Spirulina in particular, is a source of disease-fighting compounds called phytochemicals. Spirulina has been reported in the scientific literature to inhibit AIDS viral replication in human derived T-cell lines and in human peripheral blood mononuclear cells (Ayehunie S. et al. 7^{th} Conference Knysna, South Africa, Apr. 17, 1996). Spirulina extract also has been found to inhibit multiplication of Herpes simplex, influenza, mumps virus, measles virus and human cytomegalovirus in laboratory tests (Hayashi T et al. J. Natural Prod. 1996 59(1): 83-7). Spirulina or its water extracts have been shown to suppress several types of cancer. And several types of immune cells exhibit better function against environmental toxins and infectious agents. The dark blue color of spirulina is due to a blue protein phycocyanin, which stimulates the formation of new blood cells, similar to the action of the drug EPO. Because of this latter effect, spirulina may help those with radiation sickness, which depletes blood cells.

Another alga is chlorella, which contains more than 20 vitamins and minerals, including B complex, beta-carotene, vitamins C and E, iron and calcium, almost 70% protein (in the dry state) and 19 of the 22 amino acids. Its cell wall is unusual and binds heavy metals (e.g., cadmium, lead and mercury), pesticides, PCB, etc., and carries these toxins safely out of the body. In addition, chlorella produces the Chlorella Growth Factor (CGF), which is believed to improve the activity of T- and B-cells which mount the defense against viruses. It is considered to help anemia and stimulate regeneration of tissue.

Fungi also can be beneficial to human nutrition. In particular, mushrooms are popular foods and nutrition sources. Examples include but are not limited to shiitake, reishi and maitake. Shiitake is a light amber fugus whose caps have ragged gills and are covered with white flocking. Shiitake is nonpoisonous but may occasionally cause skin rashes or other allergic effects. Shiitake is widely held to fight tumors and viruses and enhance the immune system.

Reishi are shiny capped mushrooms which vary in color from reddish orange to black. Recent test tube studies have demonstrated antiallergic, anti-inflammatory, antibacterial and antioxidant effects. Reishi extracts in animal experiments have shown low toxicity. The third type of medicinal mushroom is maitake. Laboratory experiments have shown that its extract can inhibit tumor growth and stimulate the immune system of cancerous mice; Type 2 diabetes in mice was well treated by maitake.

Cactus, such as opuntia and nopalea grande, have healthful properties. Opuntia flowers (red cylinders) and pads (protruding from the plant at odd angles) are used in traditional medicine. Before use, the pads are relieved of needles and peeled. The gelatinous inside, similar to the aloe gel, has been used for adult-onset (type 2) diabetes and recently has been alleged to be useful in physical stress to increase stamina.

Fruits and vegetables are an excellent source of nutrients and may be included in the inventive composition. Examples include, but are not limited to alfalfa, apple, apricot, banana, beet, blackberry, blueberry, cantaloupe, carrot, celery, cherry, cranberry, grape, grapefruit, green barley, lemon, green lettuce, kale, kiwi, mango, orange, papaya, parsley, peach, pear, pineapple, plum, prune, raspberry, spinach, strawberry, tangerine, tomato, watermelon, wolfberry or a combination thereof. The fruit or vegetable can be provided as a juice, juice concentrate, puree or extract. The whole plant or part of the plant may be used.

Vitamins are substances that have been identified as needed by the body in micro amounts, like certain minerals. The inventive formulation is comprised of vitamin A, Vitamin C, Vitamin D, Vitamin E and the B complex vitamins. Vitamin A prevents night blindness and other eye disorders, keeps skin moist and elastic, maintains healthy hair, skin, and gums, reduces the risk of breast cancer, helps alleviate mastodynia, reduces the risk of lung cancer, maintains cell structure and integrity, works as antioxidant to prevent cell aging, helps prevent infection, and negates skin wrinkling and the effects of sun damage. Vitamin A is a fat soluble vitamin. The term vitamin A is used to include retinol and other chemically similar compounds referred to as retinoids. Beta-carotene and other carotenoids are provitamins and are only turned into retinol as the body requires. Preferably, the mixed carotenoids are lutein and zeaxanthine. Lutein and zeaxanthine have been found to decrease the risk and even reverse the development of age-related macular degeneration, the leading cause of blindness in those over the age of 65.

Preferably, in the inventive combination, vitamin A is provided in the form of beta-carotene and other mixed carotenoids. Preferably, the combination is comprised of about 5000 I.U of vitamin A. More preferably, the inventive combination is comprised of about 5000 I.U. of vitamin A in the form of natural beta-carotene.

Vitamin C, also known as ascorbic acid, is necessary for the synthesis of collagen and is used as an antioxidant. Vitamin C fights infection, reduces inflammation, heals wounds, reduces the risk of heart disease, lowers cholesterol, reduces the risk of lung, stomach, and esophageal cancers, reduces cervical epithelial abnormalities, inhibits N-nitrosamine, and reduces the severity of colds. Preferably, the inventive is comprised of at least about 50 mg, more preferably about 100 mg and most preferably about 300 mg of vitamin C.

Vitamin D is also an essential vitamin that is included in the inventive combination. Vitamin D assists in the mineralization and calcification of bone, prevents rickets in children, prevents osteomalacia in adults, preserves bone and tooth growth, and lowers blood pressure. Vitamin D is fat soluble. Preferably, the inventive combination includes at least about 100 I.U., more preferably about 200 I.U. and most preferably about 400 I.U. of vitamin D.

Vitamin E is needed for the maintenance of cell membranes and for neurological health. Vitamin E relieves hot flashes, relieves mastodynia, helps in fighting fibrocystic breast disease, reduces mammary tumors, reduces the risk of lung cancer, and reduces the risk of heart disease. Vitamin E is the generic term for a group of related substances which include alpha-tocopherol, beta-tocopherol, gamma-tocopherol, and delta-tocopherol. In addition, each of these four compounds have a “d” form, which is the natural form, and a “dl” form which is the synthetic form. Preferably, in the inventive combination, vitamin E is provided in the form of d-alpha tocopherol acetate. If the “dl” form is used, more is necessary. Preferably, the inventive combination includes at least about 20 I.U., more preferably about 40 I.U. and most preferably about 60 I.U. of vitamin E. More preferably, the inventive combination is comprised of about 60 I.U. of vitamin E in the form of d-alpha tocopherol acetate.

The inventive combination includes most of the B complex of vitamins. The B vitamins are water-soluble. The B vitamins included in the inventive combination are thiamin (vitamin B1), riboflavin (vitamin B2), niacin (vitamin B3), pantothenic acid (vitamin B5), pyridoxine (vitamin B6), biotin, folic acid, and the cobalamins (vitamin B 12).

Vitamin B 1 or thiamin helps keep collagen-rich connective and mucous membranes healthy, helps to maintain smooth muscles, helps in the formation of blood cells, and is necessary for proper nervous system function. Preferably, the inventive combination is comprised of at least 0.5 mg, more preferably at least 1.0 mg and most preferably about 1.5 mg of thiamin.

Vitamin B2 or riboflavin is necessary for healthy hair, nails, and mucous membranes and is involved in red blood cell formation, antibody production, and overall growth. Preferably, the inventive combination is comprised of at least about 0.5 mg, more preferably about 1 mg and most preferably about 1.6 mg of riboflavin.

Vitamin B3 or niacin helps in the production of most of the sex hormones, dilates blood vessels, lowers cholesterol, and helps maintain blood circulation. Niacin is the generic name for a group of compounds that exhibit niacin activity and includes niacinamide and nicotinic acid. Preferably, in the inventive combination, vitamin B3 is provided as niacinamide. Preferably, the inventive combination is comprised of at least about 5 mg, more preferably at least about 10 mg and most preferably at least about 20 mg of vitamin B3. Preferably, the multi-vitamin and mineral supplement is comprised of about 20 mg of vitamin B3 in the form of niacinamide.

Vitamin B6 or pyridoxine is involved in the production of ribonucleic acid (RNA) and deoxyribonucleic acid (DNA) and many other reactions in the body. Pyridoxine refers to and includes three different compounds: pyridoxine, pyridoxamine, and pyridoxal. Preferably, in the inventive combination, vitamin B6 is in the form of pyridoxine hydrochloride. Preferably, the inventive combination is comprised of at least about 1 mg of vitamin B6, more preferably at least about 2 mg and most preferably about 5 mg. More preferably, the inventive combination is comprised of about 5 mg of vitamin B6 in the form of pyridoxine hydrochloride.

Folic acid is essential in the production of red blood cells, the production of hormones, and the synthesis of DNA. Preferably, the inventive combination is comprised of at least about 200 mcg, more preferably at least about 400 mcg and most preferably at least about 800 mcg of folic acid.

Vitamin B12, or the cobalamins, is necessary for overall metabolism and is especially important in the function of the nervous system, metabolism of folic acid, and the production of red blood cells. There are at least three active forms of cobalamin: cyanocobalamin, hydroxocobalamin, and nitrocobalamin. Preferably, in the inventive combination, vitamin B12 is provided in the form of cyanocobalamin. Preferably, the inventive combination is comprised of about 3 mcg, more preferably at least about 6 mcg and most preferably at least about 15 mcg of vitamin B12. Preferably, the inventive combination is comprised of about 15 mcg of vitamin B 12 in the form of cyanocobalamin.

Biotin is necessary for the metabolism of carbohydrates, proteins, and fats and is needed for healthy skin and hair. Preferably, in the inventive combination, biotin is provided in the form of d-biotin. Preferably, the inventive combination is comprised of at least about 100 mcg, more preferably at least about 200 mcg and most preferably at least 300 mcg of biotin. More preferably, the multi-vitamin and mineral supplement is comprised of about 300 mcg of biotin in the form of d-biotin.

Pantothenic acid is important for the production of adrenal gland hormones, increases overall energy, and helps convert food into energy. Preferably, in the inventive combination, pantothenic acid is in the form of calcium d-pantothenate. Preferably, the inventive combination is comprised of at least about 5 mg, more preferably at least about 10 mg, and most preferably up to 40 mg of pantothenic acid. More preferably, the inventive combination is comprised of about 10 mg of pantothenic acid in the form of calcium d-pantothenate.

Also needed in small amounts are minerals. Daily multivitamin and mineral supplements frequently supply a wide variety of minerals. Some of the most common ones, with daily amounts are as follows: calcium (200 mg), iron (10-15 mg), magnesium (100 mg), potassium (80 mg), zinc (15 mg), copper (2 mg), phosphorus (48 mg), iodine (150 mcg) manganese (2 mg), chromium (150 mcg), chloride (72 mg), boron (150 mcg), nickel (5 mcg), silicon 2 mg), vanadium (10 mcg), tin and molybdenum. Two minerals which are recommended for supplementation are calcium and selenium. Obviously, calcium is needed to avoid osteoporosis. Supplementation varies from about 200 to 2000 mg.

The mineral selenium reduces the risk of heart attacks and heart disease, reduces the risk of cancer, protects against metal poisoning, and acts in synergy with vitamin E. Preferably, in the inventive combination, selenium is provided as selenium amino acid chelate. Preferably, the supplement is about 50 mcg per day, more preferably at least about 100 mcg and most preferably about 140 mcg of selenium as selenium amino acid chelate.

Two other nutrients are becoming popular: lutein and lycopene. Lutein is an antioxidant that benefits the eye, skin and cardiovascular health. Recent research shows that lutein supplementation (10 mg) may reverse symptoms of age-related macular degeneration, which gradually reduces vision. Lycopene is a strong antioxidant whose presence has been correlated with reduced cancers of the prostate, lung and stomach. Slight declines in six other cancers were also noted. Many people obtain lycopene from red fruit, such as tomatoes and watermelon.

The inventive combinations can be provided in forms that include but are not limited to tablet, capsule, soft gelatin capsule, solution, ointment, plaster, granule, lotion, aerosol, soap, cosmetic, suppository, cataplasma, cream, troche, paste, pill and injection. The oral product also can include a toothpaste, an oral cleanser, a chewing gum, energy bar, energy shake or confectionary.

The inventive combinations also can be mixed with pharmaceutically acceptable carriers such as xanthan gum polyvinylpyrrolidone and hydroxypropylcellulose; disintegrators such as calcium carboxymethylcellulose and sodium glycolate starch; diluents such as corn starch, lactose, soy bean oil, cellulose crystal and mannitol; lubricants such as magnesium stearic acid and talc; sweeteners such as sugar, fructose, sorbitol and aspartame; stabilizers such as sodium carboxymethylcellulose, α- or β-cyclodextrin, citric acid, white wax; preservatives such as potassium sorbate, methyl p-oxybenzoic acid, propyl p-oxybenzoic acid and sodium benzoate; and spice such as orange essential oil, ethyl vaseline, masking flavor, menthol and herb. These are just examples of compounds in the different categories; those skilled in the art are familiar with many different compounds which can perform likewise.

### EXAMPLES

### Example 1

The following ingredients were blended together: mangosteen juice (pulp and pericarp), Aloe vera juice and/or gel, vitamin blend, fructose, green tea, pH adjuster, preservatives (sodium benzoate and potassium), xanthan gum, selenium, and orange essential oils. The vitamin blend includes Vitamins A, C, D, E, B6 and B 12, thiamine, riboflavin, niacin, folate, biotin and pantothenic acid. The pH adjuster is malic acid, which also has a pleasant apple flavor. Other essential oils may be used instead of orange essential oil.

One fluid ounce (two tablespoons) of this combination has an ORAC value which has been measured at more than 90,000. The ORAC value can vary depending on the amounts and freshness of the components, mixing and filling procedure, etc.

### Example 2

The following ingredients were blended together: mangosteen juice and fruit, Aloe vera juice and/or gel, vitamin blend (cf. Example 1), fructose, apple juice, pear juice, grape juice, blueberry juice, raspberry juice, strawberry juice, cranberry juice, cherry juice, fruit blend, wolfberry juice, orange essential oils, raspberry flavor, carrot juice, tomato juice, beet juice, citric acid, selenium, xanthan gum and preservatives (sodium benzoate and potassium sorbate).

One fluid ounce (two tablespoons) of this combination has an ORAC value which has been measured at more than 70,000. The ORAC value can vary depending on the amounts and freshness of the components, mixing and filling procedure, etc.

Although the invention has been described in detail with reference to the presently preferred embodiments, those of ordinary skill in the art will appreciate that various modifications can be made without departing from the invention. Accordingly, the invention is defined only by the following claims.

1. A nutraceutical composition comprising mangosteen juice, mangosteen pericarp and aloe.

2. The nutraceutical composition of claim 1, wherein the composition additionally comprises at least one vitamin.

3. The nutraceutical composition of claim 2, wherein the vitamin is selected from Vitamin A, Vitamin C, Vitamin D, Vitamin E, Vitamins B6 and B12, biotin, niacin, pantothenic acid, thiamin, riboflavin, folic acid and a combination thereof.

4. The nutraceutical composition of claim 1, wherein the composition additionally comprises at least one type of alga.

5. The nutraceutical composition of claim 2 wherein the composition additionally comprises at least one type of alga.

6. The nutraceutical composition of claim 4, wherein the alga is selected from blue green alga, red alga, brown seaweed, chlorella or a combination thereof.

7. The nutraceutical composition of claim 4, wherein the alga is spirulina.

8. The nutraceutical composition of claim 1, wherein the composition additionally comprises at least one type of tea, wherein the tea is from leaves, crushed leaves, other plant parts, infusions thereof, or extractions thereof.

9. The nutraceutical composition of claim 2, wherein the composition additionally comprises at least one type of tea, wherein the tea is from leaves, crushed leaves, other plant parts, infusions thereof, or extractions thereof.

10. The nutraceutical composition of claim 8, wherein the tea is obtained from green tea leaves, black tea leaves, one or more herbs, or a combination thereof.

11. The nutraceutical composition of claim 1, further comprising an edible cactus.

12. The nutraceutical composition of claim 2, further comprising an edible cactus.

13. The nutraceutical composition of claim 11, wherein the edible cactus is an opuntia, a nopalea or a combination thereof.

14. The nutraceutical composition of claim 11, wherein the edible cactus is in the form of a leaf, fruit, juice, concentrate or extract.

15. The nutraceutical composition of claim 1, further comprising one or more fungi.

16. The nutraceutical composition of claim 2, further comprising one or more fungi.

17. The nutraceutical composition of claim 15, wherein the fungus is a mushroom.

18. The nutraceutical composition of claim 17, wherein the mushroom is maitake, shiitake, reishi, or a combination thereof.

19. The nutraceutical composition of claim 1, further comprising one or more herbs.

20. The nutraceutical composition of claim 2, further comprising one or more herbs.

21. The nutraceutical composition of claim 19, wherein the herb is selected from angelica, basil, calendula, chamomile, yarrow, coriander, feverfew, horehound, hyssop, echinacea, St. John's wort, summer savory, thyme, ginseng, kava, pennyroyal, valerian or a combination thereof.

22. The nutraceutical composition of claim 1, further comprising a fruit or vegetable.

23. The nutraceutical composition of claim 2, further comprising a fruit or vegetable.

24. The nutraceutical composition of claim 22, wherein the fruit or vegetable is provided as a juice, juice concentrate, puree or extract and comprises alfalfa, apple, apricot, banana, beet, blackberry, blueberry, cantaloupe, carrot, celery, cherry, cranberry, grape, grapefruit, green barley, lemon, green lettuce, kale, kiwi, mango, orange, papaya, parsley, peach, pear, pineapple, plum, prune, raspberry, spinach, strawberry, tangerine, tomato, watermelon, wolfberry or a combination thereof.

25. The nutraceutical composition of claim 1, additionally comprising at least one mineral.

26. The nutraceutical composition of claim 2, additionally comprising at least one mineral.

27. The nutraceutical composition of claim 25, wherein the mineral is selenium.

28. A nutraceutical composition comprising mangosteen juice, mangosteen pericarp and tea.

29. The nutraceutical composition of claim 28, wherein the composition additionally comprises at least one vitamin.

30. The nutraceutical composition of claim 29, wherein the vitamin is selected from Vitamin A, Vitamin C, Vitamin D, Vitamin E, Vitamins B6 and B12, biotin, niacin, pantothenic acid, thiamin, riboflavin, folic acid and a combination thereof.

31. The nutraceutical composition of claim 28, wherein the composition additionally comprises at least one type of alga.

32. The nutraceutical composition of claim 29, wherein the composition additionally comprises at least one type of alga.

33. The nutraceutical composition of claim 31, wherein the alga is selected from blue green alga, red alga, chlorella or a combination thereof.

34. The nutraceutical composition of claim 33, wherein the alga is spirulina.

35. The nutraceutical composition of claim 28, wherein the tea is from leaves, crushed leaves, other plant parts, infusions thereof, or extractions thereof.

36. The nutraceutical composition of claim 35, wherein the tea is obtained from green tea leaves, black tea leaves, one or more herbs or a combination thereof.

37. The nutraceutical composition of claim 28, further comprising an edible cactus.

38. The nutraceutical composition of claim 29, further comprising an edible cactus.

39. The nutraceutical composition of claim 37, wherein the edible cactus is an opuntia, a nopalea or a combination thereof.

40. The nutraceutical composition of claim 37, wherein the edible cactus is in the form of a leaf, fruit, juice, concentrate or extract.

41. The nutraceutical composition of claim 28, further comprising one or more fungi.

42. The nutraceutical composition of claim 29, further comprising one or more fungi.

43. The nutraceutical composition of claim 41, wherein the fungus is a mushroom.

44. The nutraceutical composition of claim 43, wherein the mushroom is maitake, shiitake, reishi, or a combination thereof.

45. The nutraceutical composition of claim 28, further comprising one or more herbs.

46. The nutraceutical composition of claim 29, further comprising one or more herbs.

47. The nutraceutical composition of claim 45, wherein the herb is selected from angelica, basil, calendula, chamomile, yarrow, coriander, feverfew, horehound, hyssop, echinacea, St. John's wort, summer savory, thyme, ginseng, kava, pennyroyal, valerian or a combination thereof.

48. The nutraceutical composition of claim 28, further comprising a fruit or vegetable.

49. The nutraceutical composition of claim 29, further comprising a fruit or vegetable.

50. The nutraceutical composition of claim 48, wherein the fruit or vegetable is provided as a juice, juice concentrate, puree or extract and comprises alfalfa, apple, apricot, banana, beet, blackberry, blueberry, cantaloupe, carrot, celery, cherry, cranberry, grape, grapefruit, green barley, lemon, green lettuce, kale, kiwi, mango, orange, papaya, parsley, peach, pear, pineapple, plum, prune, raspberry, spinach, strawberry, tangerine, tomato, watermelon, wolfberry or a combination thereof.

51. The nutraceutical composition of claim 28, further comprising at least one mineral.

52. The nutraceutical composition of claim 29, further comprising at least one mineral.

53. The nutraceutical composition of claim 51, wherein the mineral is selenium.

54. A nutraceutical composition comprising mangosteen juice, mangosteen pericarp, Aloe vera juice and/or gel, a vitamin blend, fructose, green tea, a pH adjuster, preservatives such as sodium benzoate and potassium, xanthan gum, selenium, and orange essential oil or other essential oils, wherein the vitamin blend comprises Vitamins A, C, D, E, B6 and B12, thiamine, riboflavin, niacin, folate, biotin and pantothenic acid, and the pH adjuster is malic acid.

55. A nutraceutical composition comprising mangosteen juice, mangosteen pericarp, Aloe vera juice and/or gel, a vitamin blend, fructose, fruit and vegetable juices, essential oils, raspberry flavor, citric acid, selenium, xanthan gum and preservatives, wherein the vitamin blend comprises Vitamins A, C, D, E, B6 and B 12, thiamine, riboflavin, niacin, folate, biotin and pantothenic acid; the preservatives are sodium benzoate and potassium sorbate; and the essential oil is orange.

56. The nutraceutical composition of claim 55, wherein the fruit and vegetable juices are selected from apple juice, pear juice, grape juice, blackberry juice, blueberry juice, raspberry juice, strawberry juice, cranberry juice, cherry juice, fruit blend, wolfberry juice, carrot juice, tomato juice, beet juice or a combination thereof.

57. A nutraceutical composition comprising mangosteen juice, mangosteen pericarp and alga

58. A nutraceutical composition comprising mangosteen juice, mangosteen pericarp and edible cactus.

59. A nutraceutical composition comprising mangosteen juice, mangosteen pericarp and fungi, particularly mushrooms.

60. A nutraceutical composition comprising mangosteen juice, mangosteen pericarp and at least one herb.

61. A nutraceutical composition comprising mangosteen juice, mangosteen pericarp and at least one vitamin.

62. A nutraceutical composition comprising mangosteen juice, mangosteen pericarp and extracts of at least one fruit or vegetable.

63. A nutraceutical composition comprising a mangosteen extract selected from whole mangosteen, mangosteen fruit juice, mangosteen pericarp, mangosteen pericarp juice or a combination thereof.

64. The nutraceutical composition of claim 63, further comprising at least one fruit or vegetable juice, puree or extract.

65. The nutraceutical composition of claim 63, further comprising an aloe product selected from whole leaf, leaf cuticle, leaf gel, an extract thereof, or a combination thereof.

66. The nutraceutical composition of claim 63, further comprising an alga such as spirulina.

67. The nutraceutical composition of claim 63, further comprising a tea.

68. The nutraceutical composition of claim 63, further comprising at least one edible cactus.

69. The nutraceutical composition of claim 63, further comprising at least one edible fungi or mushroom.

70. The nutraceutical composition of claim 63, further comprising at least one herb.

71. The nutraceutical composition of claim 63, further comprising at least one vitamin.

72. The nutraceutical composition of claim 63, further comprising at least one mineral.
